(19) United States
(12) Patent Application Publication
(10) Pub. No.: US 2024/0287448 A1
(43) Pub. Date: Aug. 29, 2024

(54) COMPOSITIONS HAVING HIGH PROTEIN CONTENT

(71) Applicant: SUPERBREWED FOOD INC., New Castle, DE (US)

(72) Inventors: Alon Karpol, Tel Mond (IL); Aharon Eyal, Jerusalem (IL); Bryan P. Tracy, Wilmington, DE (US)

(21) Appl. No.: 18/574,028

(22) PCT Filed: Jun. 21, 2022
(86) PCT No.: PCT/IB2022/055765

§ 371 Date: Dec. 24, 2023

Related U.S. Application Data
(60) Provisional application No. 63/214,442, filed on Jun. 24, 2021.

(51) Int. Cl. C12N 1/20; C12N 1/02; C12R 1/145

(52) U.S. Cl. CPC C12N 1/205; C12N 1/02; C12N 2500/32; C12N 2500/34; C12N 2500/38; C12N 2500/42; C12N 2500/72; C12R 2001/145

(57) ABSTRACT

Provided is a composition comprising a total dry weight of proteins and amino acids in of at least 80%; and wherein at least 95 wt % of said proteins have a mass of from about 70 to about 5,000 Daltons. Further provided is a growth medium comprising the composition, methods for the manufacture of the composition and uses thereof.

### CROSS-REFERENCE TO RELATED APPLICATION

The present application gains priority from U.S. Provisional application Ser. No. 63/214,442 filed Jun. 24, 2021 which is incorporated by reference as if fully set-forth herein.

### FIELD OF THE INVENTION

The present invention, in at least some embodiments, relates to compositions, and more specifically to compositions having a total dry weight of proteins and amino acids of at least 70 wt %, wherein at least 95% of the protein has a mass of from about 100 to about 5.000 Daltons.

### BACKGROUND OF THE INVENTION

Proteins are molecules essential to the structure and function of all living organisms. They are made up of chains of amino acids linked by peptide bonds and folded in a variety of complex structures. Proteins must be broken down into amino acids and peptides by hydrolysis, using strong acids, bases or proteolytic enzymes, in order to provide nutrients in forms that cells can easily use. Protein hydrolysates (also called peptones), comprising peptides and amino acids, are the result of the hydrolysis of proteins.

Protein hydrolysates are useful as a source of amino acids and nitrogen for processes such as microbial fermentation or tissue culture systems.

Protein hydrolysates may be derived from plant or animal proteins, such as from meat, casein and whey (milk proteins), gelatin, soybean, yeast, and grains.

However, significant variations in properties such as amino acid content and relative proportions, degree of hydrolysis and the like are commonly found between different batches of hydrolysates derived from the same plant or animal proteins as starting materials, which is highly problematic for industrial-scale preparation, resulting in different end products.

Furthermore, plant and animal-derived hydrolysates are often poorly defined and may include components that are unnecessary and/or undesirable, requiring complicated downstream separation processes.

There is thus a need for protein hydrolysates that are devoid of at least some of the disadvantages of the prior art.

### SUMMARY OF THE INVENTION

According to an aspect of some embodiments of the present invention, there is provided a composition comprising a total dry weight of proteins and amino acids of at least 70%, wherein at least 95 wt % of said proteins have a mass of from about 70 to about 5,000 Daltons.

According to a further aspect of some embodiments of the present invention, there is provided a method for the manufacture of the composition of claim 1, comprising culturing cells of at least one Gram positive bacterium in a fermentation medium to obtain a fermentation broth comprising a biomass; separating said biomass from said fermentation broth to obtain separated biomass; and hydrolyzing said separated biomass to obtain said composition.

According to some embodiments, there is provided a medium for growth of a cell, comprising use of the composition as disclosed herein.

According to some embodiments, there is provided a method for growth of a cell, comprising use of the composition as disclosed herein.

### DETAILED DESCRIPTION OF THE INVENTION

The present invention, in at least some embodiments thereof, relates to compositions comprising a total dry weight of proteins and amino acids of at least 70%, wherein at least 95 wt % of said proteins have a mass of from about 100 to about 5,000 Daltons.

The present inventors have surprisingly found that the compositions of the present invention provide a much higher protein content and Vitamin B12 content than similar compositions obtained from plants or yeasts.

Furthermore, production costs for the compositions of the present invention were found to be significantly lower than those for production of yeast compositions due to the lack of requirement for oxygen in the bacterial fermentation process.

Additionally, the composition of the present invention is devoid of any anti-nutritional factors (ANF), which are molecules which inhibit growth (such as phytic acid), which are present in various plant proteins, such as soy protein, wheat protein and the like. Moreover, there is no need for additional downstream processing in the preparation of the composition of the present invention, in contrast to the one or more purification steps required, for example, with soy protein as a starting material in order to obtain an 80-90% pure isolate.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “hydrolysate” refers to a product of hydrolysis of a protein and may include a peptide and/or an amino acid.

As used herein, the term “peptide” refers to a compound having at least two linked amino acid, the compound having a molecular weight of up to about 5,000 Da.

As used herein, the term “protein” is intended to include both peptides and compounds comprising linked amino acids having a molecular weight of at least 5,000 Da.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising proteins and amino acids at a total dry weight at least 70%, wherein at least 95 wt % of said proteins have a mass of from about 70 to about 5,000 Daltons.

According to some embodiments, a total dry weight of proteins and amino acids in said composition is at least 70%, about 72%, about 74%, about 76%, about 78%, about 80%, about 82%, about 84%, about 86%, about 88%, about 90%, about 92%, about 94%, about 96%, about 98% or about 100% of the total composition.

According to some embodiments, at least 95 wt %, about 96 wt %, about 97 wt %, about 98 wt %, about 99 wt % or about 100 wt % of said proteins have a mass of from about 70 to about 5,000 Daltons.

According to some embodiments, at least 95 wt % of said proteins have a mass of about 70, about 80, about 90, about 100, about 200, about 300, about 400, about 500, about 600, about 700, about 800, about 900, about 1,000, about 1,500, about 2,000, about 2,500, about 3,000, about 3,500, about 4,000, about 4,500 or about 5,000 Daltons. According to some such embodiments, at least 95 wt % of said protein has a mass of from about 70 to about 2,000 Daltons. According to some such embodiments, at least 95 wt % of said protein has a mass of from about 70 to about 1,000 Daltons.

According to some embodiments, the composition comprises at least about 20 wt % dry weight of essential amino acids. In some such embodiments, the composition comprises up to about 50 wt % dry weight of essential amino acids, such as about 20 wt %, about 25 wt %, about 30 wt %, about 35 wt %, about 40 wt %, about 45 wt % or about 50 wt %.

According to some embodiments, the composition comprises at least about 10 wt % dry weight of branched-chain amino acids. In some such embodiments, the composition comprises up to about 20 wt % dry weight of branched-chain amino acids, such as about 10 wt %, about 11 wt %, about 12 wt %, about 13 wt %, about 14 wt %, about 15 wt %, about 16 wt %, about 17 wt %, about 18 wt %, about 19 wt % or about 20 wt %.

According to some embodiments, the composition further comprises teichoic acid, in some embodiments in an amount of at least about 500 ppm. In some such embodiments, the composition comprises up to about 5,000 ppm teichoic acid, such as about 500 ppm, about 1,000 ppm, about 1,000 ppm, about 2,000 ppm, about 2,500 ppm, about 3,000 ppm, about 3,500 ppm, about 4,000 ppm, about 4,500 ppm or about 5,000 ppm.

According to some embodiments, the composition further comprises lipoteichoic acid, in some embodiments in an amount of at least about 500 ppm. In some such embodiments, the composition comprises up to about 5,000 ppm teichoic acid, such as about 500 ppm, about 1,000 ppm, about 1,000 ppm, about 2,000 ppm, about 2,500 ppm, about 3,000 ppm, about 3,500 ppm, about 4,000 ppm, about 4,500 ppm or about 5,000 ppm.

According to some embodiments, the composition further comprises peptidoglycan, in some embodiments at a concentration of at least about 0.1 wt % dry weight peptidoglycan. In some such embodiments, the concentration of peptidoglycan is up to about 10 wt % dry weight, such as about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt % or about 10 wt %.

According to some embodiments, the composition further comprises at least one selected from the group consisting of a micronutrient, an antibiotic (such as Ampicillin, Kanamycin, tetracycline, Chloramphenicol, Erythromycin, Penicillin, Gentamycin, Vancomycin, clindamycin and combinations thereof), an anti-oxidant (such as cysteine hydrochloride, sodium sulfide, sodium sulfite, sodium metabisulfite and combinations thereof) and combinations thereof.

According to some embodiments, the micronutrient is selected from the group consisting of iron, manganese, zinc, copper, selenium, molybdenum and combinations thereof.

According to some embodiments, the composition further comprises at least one selected from the group consisting of an energy source, such as a carbohydrate and/or a fat.

According to some embodiments, the composition further comprises at least one nitrogen source, such as a nitrogen source selected from the group consisting of a purine, a pyrimidine, an amino acid, peptidoglycan, ammonia, urea, nitrate, nitrite, nitrogen gas and combinations thereof.

According to some embodiments, the composition further comprises a carbon source, such as carbohydrates, fats/oil, carbon monoxide, Carbon di-oxide, carbonate, organic acids, organic solvents or combinations thereof.

According to some embodiments, the composition further comprises a growth factor. According to some such embodiments, the growth factor comprises a vitamin.

According to some embodiments, the vitamin is selected from the group consisting of folic acid, pyridoxine hydrochloride, riboflavin, biotin, thiamine, nicotinic acid, calcium pantothenate, vitamin B12, p-aminobenzoic acid, thioctic acid, hydroxamate and combinations thereof.

According to some embodiments, the composition comprises vitamin B12, in some embodiments in an amount of at least about 0.07 ppm. In some such embodiments, the composition comprises up to about to about 0.1 ppm, such as about 0.07 ppm, about 0.08 ppm, about 0.09 ppm or about 0.1 ppm.

According to some embodiments, the growth factor is selected from the group consisting of a hormone (such as insulin and/or transferrin) and/or a cytokine (such as fibroblast growth factor and/or transforming growth factor (TGF-b)).

According to some embodiments, the composition further comprises at least one trace element selected from the group consisting of, iron, zinc, copper, manganese, molybdenum, selenium and combinations thereof.

According to some embodiments, the composition further comprises at least one mineral selected from the group consisting of sodium, potassium, magnesium, calcium, phosphorus and combinations thereof.

According to some embodiments, the composition comprises a hydrolysate of a bacterium. According to some such embodiments, the hydrolysate is of at least one protein of the bacterium. According so some such embodiments, the hydrolysate of the protein provides the proteins and amino acids at a total dry weight of at least 70%, wherein at least 95 wt % of said proteins have a molecular weight of from about 70 to about 5,000 Daltons.

According to some embodiments, said bacterium is of the class Clostridia. According to some such embodiments, said bacterium is of the genus Clostridium.

According to some such embodiments, said bacterium comprises Clostridium tyrobutyricum.

According to some embodiments, the composition comprises at least about 100 ppm of at least one selected from the group consisting of butyric acid, propionic acid, deoxyribonucleic acid (DNA) from said Gram positive bacterium, ribonucleic acid (RNA) from said Gram positive bacterium, hydrolysates thereof and combinations thereof. In some such embodiments, the composition comprises up to about 10,000 ppm of at least one selected from the specified group, such as about 100 ppm, about 200 ppm, about 500 ppm, about 1,000 ppm, about 2,000 ppm, about 3,000 ppm, about 4,000 ppm, about 5,000 ppm, about 6,000 ppm, about 7,000 ppm, about 8,000 ppm, about 9,000 ppm or about 10.00 ppm.

According to some embodiments, the composition has a water content of from about 1 wt % to about 90 wt %, such as about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt % or about 90 wt % of the total composition.

According to some embodiments, the composition is in the form of a powder.

According to an aspect of some embodiments of the present invention, there is provided a method for the manufacture of the composition disclosed herein, comprising culturing cells of at least one Gram positive bacterium in a fermentation medium to obtain a fermentation broth comprising a biomass; separating said biomass from said fermentation broth to obtain separated biomass; and hydrolyzing said separated biomass to obtain said composition.

According to some embodiments, said separating comprises microfiltration.

According to some embodiments, the method further comprises drying said separated biomass to provide a dry biomass having a water content of no greater than 5 wt %.

According to some embodiments, the method further comprises milling said dry biomass to a particle size of no greater than about 2 microns, such as about 2 microns, about 1.5 microns, or about 1 micron.

According to some embodiments, hydrolyzing comprises contacting with a protease.

According to some embodiments, the protease may comprise an endopeptidase, an exopeptidase or a combination thereof. According to some embodiments, the protease may have a neutral, an acidic or an alkaline pH. According to some embodiments, the protease is selected from the group consisting of a cysteine protease, a serine protease, a metallo-protease, an aspartyl protease and a combination thereof. According to some embodiments the protease is selected from the group consisting of subtilisin A, Trypsin, Chymotrypsin, calpain, Pepsin, Cathepsin.

According to some embodiments, hydrolyzing is conducted under empirical conditions. According to an exemplary embodiment, hydrolyzing is conducted at a temperature of at least 50° C., at a enzyme: substrate weight ratio of from about 1% w/w to about 3% w/w, for a reaction time of from about 10 to about 24 hours.

According to an aspect of some embodiments of the present invention, there is provided a medium for growth of a cell, comprising the composition of any one of the embodiments disclosed herein.

According to some embodiments, the medium further comprises at least one selected from the group consisting of a macronutrient, a micronutrient, a trace element, a mineral, a buffering agent, an antifoaming agent, an antibiotic, an anti-oxidant and combinations thereof.

According to some embodiments, the buffering agent is selected from the group consisting of citric acid, acetic acid, monopotassium phosphate, N-Cyclohexyl-2-aminoethanesulfonic acid, borate, boric acid; [tris(hydroxymethyl)methylamino]propanesulfonic acid); 2-(bis(2-hydroxyethyl)amino)acetic acid); (tris(hydroxymethyl)aminomethane, or 2-amino-2-(hydroxymethyl)propane-1,3-diol); (N-[tris(hydroxymethyl)methyl]glycine); (3-[N-tris(hydroxymethyl)methylamino]-2-hydroxypropanesulfonic acid); (4-(2-hydroxyethyl)-1-piperazineethanesulfonic acid); (2-[[1,3-dihydroxy-2-(hydroxymethyl)propan-2-yl]amino]ethanesulfonic acid); (3-(N-morpholino)propanesulfonic acid); (piperazine-N,N′-bis(2-ethanesulfonic acid)); dimethylarsenic acid); (2-(N-morpholino)ethanesulfonic acid); a carbonate buffer; and combinations thereof.

According to some embodiments, at least one of the micronutrient, the antibiotic and the anti-oxidant is present in the composition prior to preparation of the final medium. In some embodiments, at least one of the micronutrient, the antibiotic and the anti-oxidant is added to the composition in order to provide the final medium.

According to some embodiments, the medium comprises at least one trace element selected from the group consisting of iron, zinc, copper, manganese, molybdenum, selenium and combinations thereof. According to some embodiments, said trace element is present in the medium at a final concentration of from about 1 to about 200 ppm, such as about 1, about 5, about 10, about 20, about 30, about 40, about 50, about 60, about 70, about 80, about 90, about 100, about 110, about 120, about 130, about 140, about 150, about 160, about 170, about 180, about 190 or about 200 ppm of the total medium.

According to some embodiments, the medium comprises at least one mineral selected from the group consisting of sodium, potassium, magnesium, calcium, phosphorus and combinations thereof. According to some of from about 1,000 to about 10,000 ppm, such as about 1,000, about 2,000, about 3,000, about 4,000, about 5,000, about 6,000, about 7,000, about 8,000, about 9,000 or about 10,000 ppm of the total medium.

According to some embodiments, the macronutrient is selected from the group consisting of phosphorus, potassium, calcium, magnesium, sulfur and combinations thereof.

According to an aspect of some embodiments, there is provided a method for growth of a cell, comprising growing said cell in the medium as disclosed herein.

According to some embodiments, the cell is a microbial cell. According to some such embodiments, the microbial cell is selected from the group consisting of a bacterial cell, a fungal cell, a protozoal cell, a viral cell, an algal cell and combinations thereof.

According to some embodiments, the bacterial cell is a probiotic bacterial cell.

According to some embodiments, the cell is a plant cell or an animal cell.

According to some embodiments, the animal cell is selected from the group consisting of a blood cell and an antibody-producing cell. According to some embodiments, the animal cell is from an immortalized cell line.

According to some such embodiments, the medium further comprises bovine serum albumin (BSA) and/or at least one hormone. According to some embodiments, the antibody-producing cell is selected from the group consisting of B-cells, T-cells, Chinese hamster ovary (CHO) cells, human colorectal adenocarcinoma cells (CACO-2) cells and combinations thereof.

According to an aspect of some embodiments, there is provided a method for culture of a tissue, comprising culturing said tissue in the medium as disclosed herein.

1. A composition comprising proteins and amino acids at a total dry weight of at least 70%, wherein at least 95 wt % of said proteins have a molecular-weight of from about 70 to about 5,000 Daltons.

2. The composition of claim 1, further comprising at least 500 ppm teichoic acid.

3. The composition of claim 1, comprising at least 500 ppm lipoteichoic acid.

4. The composition of claim 1, further comprising a dry weight at least 0.1 wt % peptidoglycan.

5. The composition of claim 1, wherein at least 95 wt % of said protein has a molecular-weight of from about 70 to about 2,000 Daltons.

6. The composition of claim 1, wherein at least 95 wt % of said protein has a molecular-weight of from about 70 to about 1,000 Daltons.

7. The composition of claim 1, further comprising at least one selected from the group consisting of a micronutrient, an antibiotic, an anti-oxidant and combinations thereof.

8. The composition of claim 1, further comprising a source of carbohydrate and/or fat.

9. The composition of claim 1, further comprising a dry weight of at least 0.07 ppm vitamin B12.

10. The composition of claim 1, comprising a dry weight of at least about 20 wt % of essential amino acids.

11. The composition of claim 1, comprising a dry weight of at least about 10 wt % of branched-chain amino acids.

12. The composition of claim 1, further comprising at least one mineral selected from the group consisting of calcium, iron, zinc, copper, manganese, molybdenum, selenium and combinations thereof.

13. The composition of claim 1, comprising a hydrolysate of a bacterium.

14. The composition of claim 13, wherein said bacterium is of the class Clostridia.

15. The composition of claim 14, wherein said bacterium is of the genus Clostridium.

16. The composition of claim 15, wherein said bacterium comprises Clostridium tyrobutyricum.

17. The composition of claim 1, comprising at least 100 ppm of at least one selected from the group consisting of butyric acid, propionic acid, deoxyribonucleic acid (DNA) from said Gram positive bacterium, ribonucleic acid (RNA) from said Gram positive bacterium, hydrolysates thereof and combinations thereof.

18. The composition of claim 1, having a water content of from about 1 wt % to about 90 wt %.

19. The composition of claim 1, in the form of a powder.

20. A method for the manufacture of the composition of claim 1, comprising culturing cells of at least one Gram positive bacterium in a fermentation medium to obtain a fermentation broth comprising a biomass; separating said biomass from said fermentation broth to obtain separated biomass; and hydrolyzing said separated biomass to obtain said composition.

21-33. (canceled)
